(19) United States
(12) Patent Application Publication
(10) Pub. No.: US 2024/0238457 A1
(43) Pub. Date: Jul. 18, 2024

(54) Gel Loaded with Alpha-Emitter Radionuclides

(71) Applicant: ALPHA TAU MEDICAL LTD., Jerusalem (IL)

(72) Inventors: Vered Domankevich, Hod Hasharon (IL); Michael Schmidt, Kfar Saba (IL); Itzhak Kelson, Tel Aviv (IL); Lior Arazi, Tel Aviv (IL); Ronen Segal, Jerusalem (IL)

(21) Appl. No.: 18/412,655

(22) Filed: Jan. 15, 2024

Related U.S. Application Data
(60) Provisional application No. 63/480,016, filed on Jan. 16, 2023.
(60) Provisional application No. 63/450,971, filed on Mar. 9, 2023.

(51) Int. Cl. A61K 51/04; A61K 9/00; A61K 9/06; A61K 33/06; A61K 47/36

(52) U.S. Cl. CPC A61K 51/0491; A61K 9/0019; A61K 9/06; A61K 33/06; A61K 47/36

(57) ABSTRACT

A mixture for treating a tumor, which includes an agent which turns into a hydrogel by addition of calcium ions, a vehicle carrying the agent in a manner allowing injection of the mixture into a tumor; and radium radionuclides bonded to the agent in a concentration sufficient to treat the tumor by radiotherapy.

### CROSS-REFERENCE TO RELATED APPLICATIONS

This application claims the benefit of U.S. provisional application 63/480,016, filed Jan. 16, 2023, and U.S. provisional application 63/450,971, filed Mar. 9, 2023, which are both incorporated herein by reference in their entirety.

### FIELD OF THE INVENTION

The present invention relates generally to tumor therapy and particularly to intra-tumoral alpha-emitter radiation therapy.

### BACKGROUND OF THE INVENTION

Ionizing radiation is commonly used in the treatment of certain types of tumors, including malignant cancerous tumors, to destroy their cells. Alpha radiation is one of the most powerful radiation types for cell destruction, but its range is very short and therefore its delivery to tumors is a challenge.

Diffusing alpha-emitters radiation therapy (DaRT), described for example in U.S. Pat. No. 8,834,837 to Kelson, mounts alpha-emitting radium radionuclides on a source (also referred to as a seed) in a manner that the radium radionuclides generally do not leave the source, but a substantial percentage of their daughter radionuclides (radon-220 in the case of radium-224 and radon-219 in the case of radium-223) leave the source into the tumor, upon radium decay. These radionuclides, and their own radioactive daughter atoms, spread around the source by diffusion up to a radial distance of a few millimeters before they decay by alpha emission. Thus, the range of destruction in the tumor is increased relative to radionuclides which remain with their daughters on the source.

In order for the treatment of a tumor to be effective, DaRT seeds employed in the treatment should be implanted throughout the tumor at small distances, e.g., less than 5 millimeters, from each other. Some tumors are easily accessible externally by a physician for implantation of the seeds, while other tumors are in internal organs. In addition, seed implantation in some organs may cause suffering and pain such as in the case of the eyeball. Even for easily accessible tumors, the destruction of a tumor may require implantation of over one hundred seeds. In addition, production of the seeds is a demanding and expensive task.

Another method used to deliver alpha emitting radioactive atoms to malignant cells is targeted radionuclide therapy using methods such as radioimmunoconjugates. In targeted therapy, targeting-carriers, such as antibodies and/or liposomes, are connected to radioactive atoms and injected into the blood stream of a patient. During circulation, the targeting-carriers attach to or remain next to malignant cells and when alpha particles are emitted by the radioactive atoms at least some of the emitted alpha particles destroy the malignant cells.

PCT publication WO01/60417 to Larsen, titled “Radioactive Therapeutic Liposomes”, PCT publication WO 02/05859 to Larsen, titled: “Method of Radiotherapy”, and US patent publication 2004/0208821 to Larsen, titled: “Method of Radiotherapy”, the disclosures of which are incorporated herein by reference in their entirety, describe liposomes which encapsulate heavy radionuclides which emit alpha particles. The radionuclides may include, among others, Radium-223, Radium-224 and Thorium-227. Daughter radionuclides generally remain trapped during nuclear transformation of the radionuclides.

PCT publication WO2006/110889, titled: “Multi-Layer Structure having a Predetermined Layer Pattern Including an Agent”, describes a polymer multilayer structure which can be used to deliver radioisotopes for radiotherapy.

Alpha emitting radioactive atoms may also be delivered to a tumor in microparticles or nanoparticles which limit the transport of the radioactive atoms into the blood stream and away from the tumor. US patent publication 2017/0000911, titled “Radiotherapeutic Particles and Suspensions”, states that the microparticles and nanoparticles may be stable or slowly degrading.

PCT publication WO2010/028048, titled: “Brachytherapy Seed with Fast Dissolving Matrix for Optimal Delivery of radionuclides to Cancer Tissue”, describes polymer seeds embedding in them microspheres containing beta-emitting or alpha emitting radionuclides. After the seeds are implanted in a tumor, they are dissolved so that the radionuclides in the microspheres can destroy tumor cells.

U.S. Pat. No. 8,470,294 describes flexible brachytherapy seeds.

U.S. Pat. No. 9,539,346 to Larsen et al. and the paper Westrøm S, Malenge M, Jorstad I S, Napoli E, Bruland ØS, Bønsdorff T B, Larsen R H., “Ra-224 labeling of calcium carbonate microparticles for internal α-therapy: Preparation, stability, and biodistribution in mice”, J Labelled Comp Radiopharm. 2018 May 30; 61(6):472-486. doi: 10.1002/jlcr.3610. Epub 2018 Mar. 12. PMID: 29380410; PMCID: PMC6001669, propose use of calcium carbonate microparticles as carriers for radium-224, designed for local therapy of disseminated cancers in cavitary regions.

US patent publication 2022/0152228 to Thorek et al., describes administration of an alpha particle emitting therapeutic agent chelating a radiotherapeutic agent with a macrocyclic molecule or combined with an ion-modulating agent, in an injectable gel or hydrogel.

U.S. Pat. No. 7,776,310 to Kaplan, titled: “Flexible and/or Elastic Brachytherapy Seed or Strand”, describes a brachytherapy strand that is elastic and/or flexible and preferably biodegradable, which carries radionuclides which may emit gamma, beta or alpha radiation. In one embodiment, the strands are hydrogel strands prepared by dripping a polymer solution, such as alginate, from a reservoir though microdroplet forming device into a stirred ionic bath.

A paper titled “Preparation of a radionuclide/gel formulation for localised radiotherapy to a wide range of organs and tissues”, by Holte et al., Pharmazie 61 (2006), describes encapsulating radio labelled particles into a gel formulation, including alginate gels.

A paper by Yu Chao et al., titled “Combined local immunostimulatory radioisotope therapy and systemic immune checkpoint blockade imparts potent antitumor responses”, Nature Biomedical Engineering, Vol. 2, August 2018, describes use of a radio-isotope-labelled natural enzyme (¹³¹I-Cat), a natural polysaccharide alginate and synthetic oligodeoxynucleotides CpG for cancer treatment.

The book “In-Situ Gelling Polymers: for Biomedical Applications” mentions various hydrogels which are used in slow release of drugs.

### SUMMARY OF THE INVENTION

There is therefore provided in accordance with embodiments of the present invention, a mixture for treating a tumor, comprising an agent which turns into a hydrogel by addition of calcium ions, a vehicle carrying the agent in a manner allowing injection of the mixture into a tumor; and radium radionuclides bonded to the agent in a concentration sufficient to treat the tumor by radiotherapy.

Optionally, the mixture has a viscosity of less than 20,000 cP. Optionally, the vehicle comprises an aqueous solution, and the agent is dispersed homogenously in the aqueous solution.

Optionally, the mixture further includes a substance which regulates immune-checkpoints dispersed in the mixture and/or a contrast material. In some embodiments, the mixture has a viscosity suitable for injection through a needle into a tumor. Optionally, the mixture is thermosensitive such that the viscosity of the mixture increases by at least a factor of two when its temperature increases from room temperature to body temperature. Optionally, the mixture is adapted to solidify less than two hours from being injected into the tumor. In some embodiments, the agent comprises alginate and/or Pluronics. In some embodiments, the radium radionuclides are radium-224 radionuclides. Optionally, the mixture further comprises calcium. Optionally, the mixture comprises calcium at a concentration of between about 5-10 millimolar. In some embodiments, the mixture does not bond to radon and lead. Optionally, the agent which turns into a hydrogel by addition of calcium ions is between 2-4% of the mixture.

There is further provided in accordance with embodiments of the present invention, a method for treating a tumor, comprising injecting into the tumor a mixture including an agent which turns into a hydrogel in contact with calcium ions; and injecting into the tumor radium radionuclides of an activity suitable for treatment of the tumor. In some embodiments, the method includes injecting calcium into the tumor. Optionally, injecting the calcium into the tumor is performed before injecting the mixture. Alternatively or additionally, injecting the calcium into the tumor is performed after injecting the mixture into the tumor. In some embodiments, injecting the calcium into the tumor comprises including calcium in the mixture and injecting the mixture into the tumor. Optionally, injecting the calcium into the tumor comprises concurrently injecting the mixture and the calcium. In some embodiments, injecting the radium radionuclides into the tumor comprises including radium in the mixture and injecting the mixture into the tumor.

There is further provided in accordance with embodiments of the present invention, a method for generating a mixture for treating a tumor, comprising providing an inert excipient, adding an agent which turns into a hydrogel in contact with calcium ions to the inert excipient; and adding radium radionuclides in a concentration sufficient to treat the tumor by radiotherapy, to the inert excipient.

Optionally, adding the agent and the radium to the inert excipient comprises combining the agent and the radium before they are added to the inert excipient, and adding the combination of the agent and the radium together to the inert excipient. Alternatively or additionally, adding the agent and the radium to the inert excipient comprises adding the radium to the inert excipient only after the agent is added to the inert excipient.

### BRIEF DESCRIPTION OF THE DRAWINGS

FIG. 1 is a flowchart of acts performed in producing a mixture for delivery of radium to a tumor, in accordance with an embodiment of the present invention; and

FIG. 2 is a flowchart of acts performed in treatment of a tumor, in accordance with an embodiment of the present invention.

### DETAILED DESCRIPTION OF EMBODIMENTS

An aspect of some embodiments of the invention relates to delivering alpha-emitter radium radionuclides to a tumor, tumor bed, or other site requiring treatment, in a mixture (e.g., solution) which comprises an agent which turns into a hydrogel by addition of calcium ions. In some embodiments, the mixture comprises calcium ions and/or other ions with properties similar to calcium, which can turn the liquid mixture into a hydrogel. The calcium and/or other ions optionally crosslink polymer chains of the agent to form a polymeric net. This may occur, for example, in a manner similar to the “egg-box” model described in “A Study of Sodium Alginate and Calcium Chloride Interaction Through Films for Intervertebral Disc Regeneration Uses”, Congresso Brasileiro de Engenharia e Ciência dos Materiais 09 a 13 de Novembro de 2014, the disclosure of which is incorporated herein by reference.

Use of such a mixture has the advantage that the agent chemically bonds to the radium, which is similar in some chemical properties to calcium, basically preventing the radium from leaving the mixture, while the mixture remains in the tumor for a sufficient time required for the treatment, due to gelation. On the other hand, the agent does not substantially bond to descendant radionuclides of the radium, such as radon and lead, which are allowed to diffuse or otherwise disperse throughout a tumor in which the mixture is implanted, despite the presence of the mixture in the tumor. The mixture is optionally injectable.

### Composition

The mixture optionally comprises in a vehicle, an agent which turns into a hydrogel when forming contact with calcium ions, and radium radionuclides which couple to the agent. In some embodiments, the vehicle comprises an inert excipient, such as water, saline and/or phosphate-buffered saline (PBS). In some embodiments, the mixture includes one or more additional drugs which are to be delivered with the radionuclides. In some embodiments, the mixture includes one or more other materials, such as a contrast material or isotope used for imaging.

The mixture optionally comprises a biocompatible aquatic solution, which has a viscosity suitable for direct injection into a tumor. The aquatic solution optionally has a viscosity of at least 10, 20, 50 or even 200 centipoise (cP), but lower than 1,000 cP, at 20 degrees Celsius. Alternatively, the aquatic solution has a high viscosity of at least 2,000 cP, at least 5,000 cP, or even at least 10,000 cP. Optionally, the required viscosity is achieved by adding a sufficient amount of calcium to the mixture. Generally, the more calcium added to the mixture, the higher the viscosity of the mixture. Alternatively, other materials may be added to the mixture to control its viscosity.

In some embodiments, the mixture is designed to spread within a tumor to which it is delivered, but to be sufficiently viscous to remain in the tumor and hold the radium within the tumor until at least 80%, at least 90%, at least 95% or even at least 97% of the radium radionuclides have undergone radioactive decay. Optionally, after injection, the mixture is sufficiently viscous such that 24 hours after delivery into a tumor, not more than 50%, not more than 30%, not more than 10%, not more than 5% or even not more than 3% of the agent, leaves the tumor.

In some embodiments, the components of the mixture that hold the radium in the tumor are not biodegradable, or are biodegradable but are only slowly degradable or begin to degrade only a predetermined period after injection, such that not more than 50%, not more than 40%, not more than 25%, not more than 15%, not more than 5%, not more than 3% or even not more than 1% of the radium that did not undergo radioactive decay is allowed to escape the tumor.

Alternatively or additionally, part of the components of the mixture not required to hold the radium in the tumor, are biodegradable in order to allow for closer and direct contact between the radium and tumor cells.

### Preparation

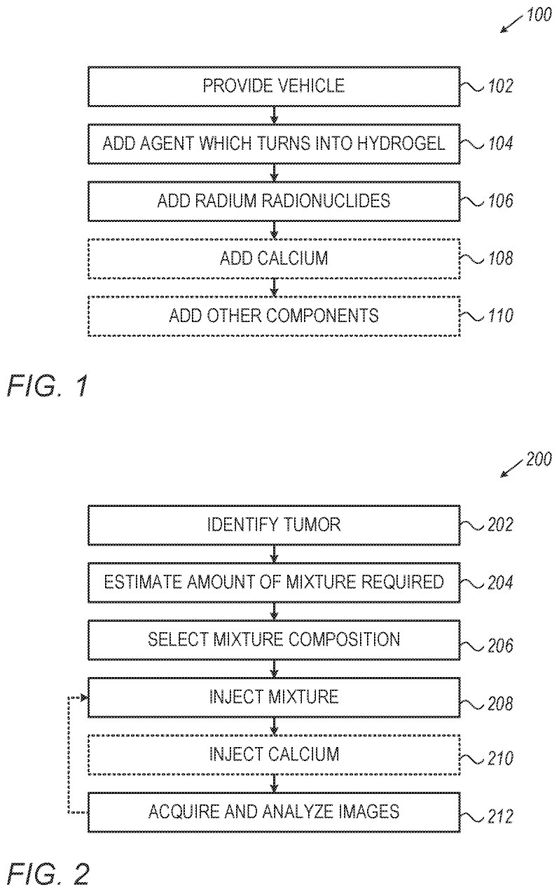

FIG. 1 is a flowchart of acts performed in producing a mixture for delivery of radium to a tumor, in accordance with an embodiment of the present invention. The method (100)

includes providing (102) a vehicle, and adding (104) to the vehicle, the agent which turns into a hydrogel when forming contact with calcium ions. In some embodiments, the vehicle serves as a diluter of the agent. Alternatively or additionally, small particles which comprise the agent are dispersed in the vehicle. Radionuclides of radium are added (106) to the agent, before or after the agent is added to the vehicle. In some embodiments, calcium is added (108) to the mixture. Alternatively or additionally, other components are added (110) to the mixture, such as one or more therapeutic drugs, an in-situ gelling polymer and/or contrast materials. In some embodiments, some or all of the components of the mixture are each provided in a separate solution, and the solutions are combined to form the mixture. In some embodiments, a first solution of 10% sodium alginate (also known as alginate) and a second solution of radium-224 radionuclides are prepared separately. The first and second solution optionally have the same size (e.g., 100 microliter each). The first and second solutions are then mixed together, such that the alginate concentration goes down to 5%.

It is noted that the method of FIG. 1 is just one example of the methods that may be used to create the mixture. Particularly, the components of the mixture may be combined in any suitable order. For example, in other embodiments, the radium is added to the vehicle before the agent is added to the vehicle. In some embodiments, the calcium is added to the vehicle before the radium and/or the agent. In other embodiments, the agent and calcium are mixed, or an agent solution and calcium solution are mixed together, and radium is added only thereafter. In still other embodiments, the calcium and radium, or solutions thereof, are mixed together, and the combined radium and calcium are mixed with the agent. Optionally, after adding the radium to the mixture, the mixture is left for an incubating period of at least 45 seconds, at least 90 seconds, at least 210 seconds, at least 360 seconds or even at least 10 minutes, in which the radium is allowed to spread and/or couple to the agent. In some embodiments the mixture is stirred or otherwise mixed to achieve a more homogeneous spread of its components. In some embodiments the mixture is sterilized before delivery to the tumor, for example using autoclave sterilization. Optionally, the sterilization is performed after the components are mixed. Alternatively, the components are sterilized separately. The mixing is optionally performed at room temperature.

In some embodiments, the mixture is prepared by adding radium radionuclides to an alginate-based product available commercially, such as GUARDIX® SG.

### The Agent

Referring in more detail to adding (104) the agent, the agent optionally comprises sodium alginate, such as described in Abasalizadeh, F., Moghaddam, S. V., Alizadeh, E. et U Alginate-based hydrogels as drug delivery vehicles in cancer treatment and their applications in wound dressing and 3D bioprinting. J Biol Eng 14, 8 (2020), doi.org/10.1186/s13036-020-0227-7, which is incorporated herein by reference. Alternatively or additionally, the agent comprises poloxamer, also known as Pluronics.

Optionally, the agent is not biodegradable, or is slowly biodegradable such that substantial degradation begins only after at least one, at least 2 or even after at least 5 half-lives of the radium radionuclides. For radium-224, the substantial degradation of the agent optionally begins only after at least 10 days, at least 20 days or even at least a month from implantation. The degradation to a level of less than 10% optionally continues over a period of at least 10 days, at least 20 days or even at least 30 days. Thus, the agent fixes the radium in place as long as it has substantial radioactivity.

In some embodiments, the agent is at least 0.1%, at least 0.4%, at least 0.5%, at least 1%, at least 2%, at least 3.5% or even at least 4.5% of the mixture in weight/weight. In some embodiments, the agent is less than 12%, less than 10%, less than 8%, less than 6.5%, less than 5% or even less than 4% of the mixture in weight/weight.

In embodiments in which the mixture includes low levels of calcium (e.g., less than 3 or even less than 2 milli-molar) in the mixture or even no calcium at all, such that the gelation is mainly due to endogenous calcium and/or other ions, the agent is optionally included in the mixture in a concentration of at least 3 mg/ml, or even at least 5 mg/ml. Optionally, the agent is included in a concentration of less than 7 mg/ml or even less than 6 mg/ml so that the mixture assumes a weak mechanical strength gel, which semi-uniformly disperses in the tumor, so that the radium in the mixture is distributed throughout the tumor, without substantial leakage outside of the tumor.

In some embodiments, the agent is included in a concentration of at least 5 mg/ml, 7 mg/ml, at least 9 mg/ml or even at least 12 mg/ml so that the mixture assumes a more viscous solid and greater-mechanical-strength gel form.

For a mixture with higher levels of calcium, such as more than 4 or even more than 5 milli-molar, the agent concentration is optionally less than 6 mg/ml, less than 5 mg/ml, less than 4 mg/ml or even less than 2 mg/ml.

In some embodiments, the agent is dispersed homogeneously in the vehicle. Accordingly, the radium, which couples to the agent, is dispersed homogeneously in the mixture. In other embodiments, the mixture is non-homogenous, including small particles (e.g., microparticles, nanoparticles and/or beads) which are formed from the agent, carry the agent or carry at least a substantial percentage of the agent. The non-homogenous structure increases the surface area of the agent from which radon can escape. As the non-agent portions of the mixture dissolve, the uniformity of the spread of the agent to which the radium is bonded increases. In still other embodiments, some (e.g., at least 20%, at least 40% or even at least 60%) of the agent in the mixture is dispersed throughout the vehicle, while another portion of the agent (e.g., at least 25%, at least 35% or at least 45%) is included in small particles.

In the present application, the term “microparticles” refers to particles having a diameter of between 0.1 and 100 micrometers. The term “nanoparticles” refers to particles having a diameter of between 0.1 and 100 nanometers. It is noted that in some embodiments the small particles are larger than microparticles, for example having a diameter of at least 150 micrometers, at least 250 micrometers or even at least 400 micrometers. Optionally, in these embodiments, the small particles have a diameter of less than 500 micrometers. In some embodiments, the small particles are spheres and/or beads. Alternatively, the small particles are of any other suitable shape.

In some embodiments, the small particles are formed by dripping a solution of the agent into a high-concentration calcium solution. In other embodiments, the small particles are produced by adding air to a solution of the agent and calcium. Alternatively or additionally, the small particles are formed by mixing the agent along with an aquatic solution. In some embodiments, the small particles are created by changing the relation between the agent and the calcium such that the resultant mixture includes small particles in an aquatic solution. The small particles are produced using any suitable method known in the art, such as any of the methods in Andrea Dodero et al., “An Up-To-Date Review on Alginate Nanoparticles and Nanofibers for Biomedical and Pharmaceutical Applications”, Advanced Materials Interfaces, Vol. 8, Issue 22, Nov. 23, 2021, Patricia Severino, “Alginate Nanoparticles for Drug Delivery and Targeting”, Current Pharmaceutical Design, Volume 25, issue 11, 2019, Anna Letocha, “Preparation and Characteristics of Alginate Microparticles for Food, Pharmaceutical and Cosmetic Application”, Polymers 2022, and/or Jerome P. Paques, “Formation of Alginate nanospheres”, Thesis, Wageningen University, 2014.

The small particles may be of any suitable type known in the art, such as microfluidic devices, e.g. those provided by CD Bioparticles (www.cd-bioparticles.net/alginates), Elve Flow (https://www.elveflow.com/microfluidics-application-packs/nanoparticles-packs/easy-microfluidic-alginate-beads-generation-pack) and/or Thomas Scientific (www.thomassci.com/nav/cat1/alginatechitinbeads/0). Methods for production of small particles, which may be used in the present invention, are described, for example, in Alginate particle production, by Elve Flow www.techusci.com/UploadFiles/2021-03/369/2021032014063196895.pdf and/or Alginate Microbeads Production, by Fluigent (www.fluigent.com/wp-content/uploads/2022/01/alginate-beads-production-application-note.pdf).

In some embodiments, the small particles are formed from a solution which already includes the radium radionuclides. In other embodiments, the small particles are formed from an inert solution which does not include the radium radionuclides and the radium radionuclides are added to the small particles after their production, for example by incubation of the small particles in a radium solution.

Optionally, the small particles are formed such that the agent and bonded radium are on the outer surface of the small particles or close to the outer surface, in a manner that daughter radionuclides resulting from radioactive decay have a high probability, e.g., at least 25%, at least 35% or even at least 40%, of leaving the small particle. For example, when adding the radium to the small particles after their production, the addition is optionally performed such that the radium concentrates on the outer surfaces of the small particles, and not in their interior. Alternatively or additionally, the small particles are produced such that their interior is formed of a material which does not attract radium, while their outer surface includes a material which attracts radium. Alternatively, in some or all of the small particles, particularly nanoparticles which are small enough to allow diffusion of radon out of the nanoparticles even from their interior, the agent and radium are in the interior of the microparticle.

The small particles optionally have a viscosity of at least 100 cP, at least 200 cP, at least 300 cP or even at least 500 cP. Optionally, the viscosity of the particles is lower than 2,000 cP, lower than 1,500 cP or even lower than 1,000 cP.

### Radium

In some embodiments, the radium ions are not coupled to large particles that are made from materials other than the agent. Specifically, the radionuclides of radium are optionally not coupled to particles having a diameter greater than 100 nanometers, except for the agent, not coupled to targeting elements (e.g., antibodies) which couple to cells, not coupled to proteins and/or enzymes (e.g., catalase) and/or not coupled to vectors which are internalized into cancer cells (e.g., liposomes, radionucleotides). Optionally, the mixture does not include large microparticles, enzymes, liposomes, radionucleotides and/or targeting elements, which may couple to the radium. Thus, the movement of the radium is constrained by the agent, which prevents the radium from leaving a tumor in which it is located, but may allow small movements within the tumor, in a manner achieving better coverage of the tumor by the radiation from the radium. In other embodiments, however, the radium is coupled to large particles and the dispersion of the radium throughout the tumor is achieved by the initial injection of the mixture throughout the tumor.

The radium optionally includes radium-224 or radium-223. In some embodiments, the mixture includes at least 2 radium molecules per 1×10¹⁰ agent molecules, at least 5 radium molecules per 1×10¹⁰ agent molecules, at least 10 radium molecules per 1×10¹⁰ agent molecules, or even at least 20 radium molecules per 1×10¹⁰ agent molecules. Optionally, however, the mixture includes less than 100 radium molecules per 1×10¹⁰ agent molecules, less than 50 radium molecules per 1×10¹⁰ agent molecules, less than 30 radium molecules per 1×10¹⁰ agent molecules, or even less than 20 radium molecules per 1×10¹⁰ agent molecules. The radium activity per volume is optionally at least 25 kilobecquerel per milliliter of the mixture, at least 75 kilobecquerel per milliliter of the mixture, at least 150 kilobecquerel per milliliter of the mixture, at least 300 kilobecquerel per milliliter of the mixture, or even at least 500 kilobecquerel per milliliter of the mixture. On the other hand, in some embodiments, the radium concentration is less than 1 megabecquerel per milliliter of the mixture, less than 700 kilobecquerel per milliliter of the mixture, less than 500 kilobecquerel per milliliter of the mixture, or even less than 400 kilobecquerel per milliliter of the mixture. The radium activity used optionally depends on the specific tumor type, according to the required biological effective dose (BED) of the tumor type and other properties of the tumor. Higher levels of activity are optionally used when a relatively large percentage of the mixture is expected to escape from the tumor.

The radium is optionally bonded to the agent. In some embodiments, after gelation, the radium is basically trapped in the mixture and the rate of radium atom release is less than 3% per day, less than 2% per day, or even less than 1% per day, during a period of at least 5 days or even at least 10 days from gelation. In some embodiments, the low leakage of radium from the mixture persists over a period of at least one half-life of the radium, at least 2 half-lives of the radium or even over at least three half-lives of the radium.

The diffusion of the radium, e.g., radium 224, in the mixture of the present embodiments is optionally very low, so that the radium does not escape the tumor in meaningful amounts. Optionally, the radium has a diffusion coefficient in the mixture of less than 10⁻¹² cm²/sec or even less than 2*10⁻¹³ cm²/sec.

On the other hand, upon radioactive decay of the radium, its daughter radionuclides (including radon and descendants thereof), have a substantial probability of leaving the mixture, for example with a release probability of at least 15%, at least 30% or even at least 40%. The radon daughter radionuclides of the radium radionuclide atoms optionally have a diffusion coefficient in the mixture greater than 10⁻⁷ cm²/sec.

In some embodiments, adding (106) the radium to the mixture is performed by first combining the radium to the agent and then adding the combined radium and agent to the vehicle. In other embodiments, the agent is first added to the vehicle and the radium is then added to the mixture of the agent and the vehicle. In further embodiments, the radium and the agent are first each placed in a separate solution, and these solutions are combined to form the mixture. In still other embodiments, the radium is first added to a calcium mixture or solution and then the combined radium and calcium are added to the mixture of the agent and the vehicle.

Optionally, adding (106) the radium is performed by adding a solution containing radium to the other components of the mixture. The solution containing radium may be generated using any suitable method known in the art, such as any of the methods described in PCT publication WO 2021/070029 “Wet preparation of Radiotherapy Sources”, the disclosure of which is incorporated herein by reference in its entirety. Alternatively or additionally, any of the methods described in Russian patent 2734429, U.S. Pat. No. 6,126,909 to Rotmensch et al., and/or U.S. Pat. No. 5,038,046 to Norman et al, the disclosures of which are incorporated herein by reference, are used to generate the solution with radium.

Alternatively, the radium is added (106) to the mixture, by placing the other components of the mixture in a flux of radium radionuclides. The flux is optionally generated by a flux generating surface source. For example, when the radionuclide is Ra-224, a flux thereof can be generated by a surface source of thorium-228 (Th-228). A surface source of Th-228 can be prepared, for example, by collecting Th-228 atoms emitted from a parent surface source of U-232. Such parent surface source can be prepared, for example, by spreading a thin layer of acid containing U-232 on a metal. Alternatively or additionally, the flux is generated using any of the methods described in US patent publication 2015/0104560 to Kelson et al., titled: “Method and Device for Radiotherapy”, the disclosure of which is incorporated herein by reference in its entirety.

Alternatively, dissolvable seeds carrying the alpha-emitter radionuclides are first generated, and the seeds are dissolved into other components of the mixture.

### Calcium

In some embodiments, the mixture includes calcium ions so that the gelation of the mixture within the tumor does not depend solely on calcium in the tumor.

Optionally, the calcium is added as free calcium ions. In some embodiments, a chloride compound is dissolved in a solution to create chloride ions, and the solution with the chloride ions is included in the mixture. The chloride compound optionally includes calcium chloride. Although other chloride compounds may also be used, such as calcium nitrate, calcium acetate and/or calcium gluconate.

In some embodiments, the calcium is added (108) to the mixture well before the injection of the mixture into the tumor. Optionally, in accordance with these embodiments, the calcium in the mixture is of a low concentration, which does not cause the mixture to turn into a gel on its own, but makes the gelation occur faster upon injection, and/or makes the viscosity of the mixture after gelation higher than when depending only on calcium in the tumor. In accordance with this option, the calcium is optionally provided in stand-alone calcium ions. In other embodiments, the calcium is included in calcium-loaded nanoparticles, such as liposomes. In some of these embodiments, the calcium-loaded nanoparticles are thermosensitive and set to release the calcium in body temperature or close to body temperature (e.g., from above 32° C., above 33° C., above 34° C.), so the agent turns into a gel after being inserted into the tumor. Alternatively or additionally, the calcium-loaded nanoparticles are designed to release their calcium upon injection due to other reasons, such as pH differences. In still other embodiments in which calcium is added (108) to the mixture sufficiently before the injection into the patient, the added calcium causes gelation to occur before the injection and the mixture is injected in a gel form.

In other embodiments, the calcium is added (108) to the mixture shortly before the injection of the mixture into the tumor, and causes complete gelation or solidification only after several seconds or minutes, so that gelation and/or solidification of the mixture occurs only after injection.

Optionally, the calcium is added (108) to the mixture only after the radium is added (106) and bonded to the agent, such that the calcium does not interfere with bonding the radium to the agent. Alternatively, the calcium is added (108) to the mixture before the radium is added (106). In accordance with this alternative, the calcium in the mixture is optionally less than covers the entire agent, so as to allow the radium to bond to the agent.

The concentration of calcium in the mixture before injection governs the percentage of radium that remains in the tumor over time. A low concentration of calcium leads to a mixture that has a low viscosity, and hence higher levels of radium leave the tumor before radioactive decay. A high concentration of calcium may lead to too high a viscosity of the mixture at the time of injection to the tumor, such that the mixture is not injectable or does not disperse properly within the tumor.

Optionally, the calcium is at least 0.1%, at least 0.3%, at least 0.5%, at least 0.8%, at least 1.5% or even at least 2.5% of the mixture in weight/weight (w/w). In some embodiments, the calcium is less than 10%, less than 8%, less than 6%, less than 5%, or even less than 4% of the mixture w/w. In some embodiments, for example embodiments in which the calcium in the mixture is not intended to cause the mixture to turn into a gel on its own, the calcium is added (108) at a concentration of at least 1 millimolar (mM), at least 2 mM, at least 4 mM, at least 6 mM or even at least 8 mM, but at a concentration of less than 30 mM, less than 20 mM, less than 15 mM, or even less than 12 mM. In other embodiments, for example embodiments in which the mixture is intended to be delivered to the treatment site as a gel, the calcium in the mixture is of a concentration of at least 50 millimolar, at least 75 millimolar, or even at least 100 millimolar.

Alternatively, the mixture does not include calcium, and the agent turns into a hydrogel within the tumor by collecting endogenous calcium present in the tumor after administering the mixture or by collecting calcium injected to the tumor separately, before, concurrently with or after injecting the mixture. Optionally, the calcium is injected to the tumor before, during and/or after injection of the mixture, through a separate needle, different from the needle used for injection of the mixture. In some embodiments, the calcium is injected concurrently with the mixture, for example using a split needle. In some embodiments, the mixture is designed to have a low pH value in order to extract calcium from surrounding tissue.

In still other embodiments, instead of using calcium to induce gelation of the agent, any other suitable component which hardens the agent is used, such as any bio-compatible element of the elements mentioned as inducing gelation of alginate in Hu et al., “Ions-induced gelation of alginate: Mechanisms and Applications”, International Journal of Biological Macromolecules, the disclosure of which is incorporated herein by reference. For example, instead of using calcium for gelation, magnesium is used to induce the gelation.

### Further Components

In some embodiments in which the radium is included in small particles, the small particles are targeted by adding antibodies thereto. Optionally, additional materials are added in order to help in linking to antibodies for targeting the particles to the tumor.

Optionally, the mixture includes a contrast material, such as gold, titanium, titanium oxide, zirconium oxide, silicon oxide, and/or any other suitable metal, which clearly appear in an imaging modality. In some embodiments, the contrast material comprises radionuclides other than radium, which emit alpha, beta and/or gamma radiation to be used primarily for imaging. Optionally, after injecting the mixture into a tumor, the tumor may be imaged to determine a layout of the mixture in the tumor and whether further injection of the mixture is required.

In some embodiments in which the agent is included in small particles, the contrast material is included in the small particles. In some embodiments, the contrast material is mixed with the agent and the small particles are formed from the mixture of the contrast material and the agent. Alternatively or additionally, the small particles include a metal base, e.g., a gold base, coated by the agent, or by the agent with the inert excipient.

Optionally, in order to prevent sodium ions in the patient from detaching the calcium formed bonds generating the gel, the mixture includes aldehyde, which prevents the sodium from detaching the bonds of the hydrogel.

The one or more additional materials includes, for example, any of the materials listed in Andrea Dodero et al., “An Up-to-Date Review on Alginate Nanoparticles and Nanofibers for Biomedical and Pharmaceutical Applications”, 2021, available at doi.org/10.1002/admi.202100809, the disclosure of which is incorporated herein by reference.

In some embodiments, the one or more additional materials do not substantially prevent diffusion of radium daughters (including daughters of daughters down a decay chain). That is, the one or more additional materials are optionally chosen as materials which do not couple to and/or block diffusion of progeny of radon, such as lead.

Further alternatively or additionally, the vehicle comprises an in-situ gelling polymer, which is supplied in a sol form at room temperature and upon injection into a patient transforms into a gel state, in response to a change in temperature, pH and/or ionic composition. For example, any suitable in-situ gelling polymer described in Kouchak M. In situ gelling systems for drug delivery, Jundishapur J Nat Pharm Prod. 2014 Jun. 1; 9(3):e20126. doi: 10,17795/jjnpp-20126. PMID: 25237648; PMCID: PMC4165193 and/or in Xian Jun Loh, “In-Situ Gelling Polymers, for Biomedical Applications”, 2014, the disclosures of which are incorporated herein by reference.

Combination with Other Drugs

In some embodiments, in addition to alpha-emitter radium radionuclides, the mixture includes one or more drugs. The one or more drugs may be any drugs suitable for treatment of the patient, for example drugs known to have a positive synergy with alpha-emitter radiation treatment. In some embodiments, the one or more drugs include a substance which activates cytoplasmatic sensors for intracellular pathogen in the tumor, as described for example in PCT publication WO 2020/089819, titled: “Intratumoral Alpha-Emitter Radiation and Activation of Cytoplasmatic Sensors For Intracellular Pathogen”, the disclosure of which is incorporated herein by reference in its entirety. Alternatively or additionally, the one or more drugs include an immune checkpoint regulator, such as described in PCT application PCT/IB2022/055680, titled: “Intratumoral Alpha-Emitter Radiation in Combination with Checkpoint Regulators”, the disclosure of which is incorporated herein by reference in its entirety. Further alternatively or additionally, the one or more drugs include a vasculature inhibitor, such as described in PCT application PCT/IB2022/055679, titled: “Intratumoral Alpha-Emitter Radiation in Combination with Vasculature Inhibitors”, the disclosure of which is incorporated herein by reference in its entirety. In some embodiments, the one or more drugs included in the mixture comprise immunoadjuvants, such as TLR agonists and/or PolyIC, Alternatively or additionally, the one or more drugs included in the mixture comprise antibodies such as antiangiogenic agents and/or immune blockades.

Alternatively or additionally, the one or more drugs include any other suitable drug for chemotherapy, immunotherapy (e.g., aPD-1), Gene therapy, targeted therapy and/or antiangiogenic treatment. The one or more drugs may be dispersed throughout the mixture and/or may be placed in the small particles.

### Combined Treatment

Optionally the mixture can be injected to a patient in combination with one or more other anticancer treatments such as radiation, immunotherapy, targeted therapy, chemotherapy and surgery. Optionally these treatments synergize with alpha-emitters (refer to the previous patents). Optionally the other anticancer treatments be given before, concurrently or after the injection of the mixture.

### Experiment

Applicant has performed an experiment in which a radium water-based solution was mixed with sodium alginate solution such that the final concentration of sodium alginate in the solution was 4% w/w. The resultant solution was mixed with a calcium chloride water-based solution in the concentration of 1% w/w. The resultant hydrogel was then placed in fetal bovine serum. The serum was replaced by a clean serum solution every 3 days for 4 times, and the replaced serum was subjected to activity measurements that characterized the Probability of Pb-212 release from the gel to the serum (%) and the rate of radium leakage from gel per day (%), for each timepoint.

The results for each timepoint are presented in the following table:

| | | | Ra-224 leakage |
|---|---|---|---|
| | Probability of Pb-212 | Ra-224 leakage | from gel, per |
| Day | release from gel, % | from gel, % | day % |
| day 3 | 41% | 5% | 1.7% |
| day 6 | 46% | 3% | 1.0% |
| day 9 | 30% | 3% | 1.0% |
| day 12 | 24% | 2% | 0.7% |

The experiment shows that Ra-224 molecules are relatively highly fixated to alginate-based hydrogel, while radium daughter atoms are released from the gel to the outer liquid environment, in substantial amounts. Other gels may allow leakage of larger percentages of radium or have a much lower release rate of daughter radionuclides.

### Usage

Exemplary tumors that can be treated by the mixture include tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma. In some embodiments, the mixture is used to treat eye cancer, e.g., uveal melanoma.

### Administration

FIG. 2 is a flowchart of acts performed in treatment of a tumor, in accordance with an embodiment of the present invention. The method (200) optionally includes identifying (202) a tumor or other tissue requiring treatment and estimating (204) an amount of the mixture required to be injected and/or selecting (206) the precise composition of the mixture to be injected to the identified tumor. Thereafter, the mixture is injected (208) to the tumor. In some embodiments, calcium is injected (210) to the tumor before, during and/or after injection of the mixture. Optionally, a medical image of the tumor is acquired and analyzed (212), to determine whether the entire volume of the tumor is covered by the mixture. If necessary, a further injection (208) of the mixture to uncovered areas of the tumor is carried out.

Referring to estimating (204) of the amount of the mixture to be injected, the amount is optionally selected proportionally to the size of the tumor. In some embodiments, the amount of the mixture injected to a tumor is at least 3%, at least 5% or even at least 10% of the volume of the tumor. The injected mixture is optionally of a volume of less than 50%, less than 25%, less than 20% or even less than 15% of the volume of the tumor.

In some embodiments the mixture is injectable and/or non-solid. In some of these embodiments, the mixture is injected as a liquid. Optionally, the mixture when injected has a viscosity lower than 25 millipascal seconds, lower than 20 millipascal seconds, lower than 15 millipascal seconds, or even lower than 10 millipascal seconds. Alternatively, the mixture has a gel structure when being injected. At the time of injection, the mixture optionally has a viscosity of less than 10,000 centipoise (cP), less than 5,000 cP or even less than 3,000 cP, so as to allow easy injection to a tumor.

Optionally, inside the tumor after being injected, the viscosity of the mixture increases, and possibly even solidifies. Optionally, the viscosity of the mixture is adjusted by changing its pH. In some embodiments, the mixture is thermosensitive. At room temperature (e.g., 21° C.) or lower, the mixture optionally has a viscosity of less than 10,000 centipoise (cP), less than 5,000 cP or even less than 3,000 cP, while in body temperature the mixture has a high viscosity of at least 75,000 cP, at least 85,000 cP, at least 90,000 cP or even at least 95,000 cP. Further alternatively, the viscosity of the mixture increases in the tumor due to its exposure to endogenous calcium ions. In other embodiments, however, at the time of injection the mixture has a high viscosity of at least 20,000 cP, at least 40,000 cP or even at least 70,000 cP, so that the mixture substantially remains close to the point in which it was injected.

In other embodiments, the mixture solidifies before being injected and is introduced into the tumor in the form of flexible seeds of any suitable shape. In still other embodiments, the mixture is included as an outer layer on a seed, catheter or needle inserted into the tumor. Optionally in these embodiments, the seed is covered by a protective layer which protects the mixture from being removed from the seed before insertion into the tumor.

In some embodiments, the mixture is injected (208) to a core of the tumor. Alternatively or additionally, the mixture is injected to margins of the tumor and/or adjacent the tumor outside of the tumor, for example when treating remnants of a tumor after removal by surgery. In some embodiments, the mixture is injected throughout the tumor. In some embodiments, a needle tip through which the mixture is injected is moved, e.g., retracted, during the injection in order to increase the surface area of the mixture in the tumor, and thus increase the volume of the tumor effected by the radium. The mixture is optionally injected to the tumor in a single insertion of a needle. Alternatively, the mixture is injected to the tumor in a plurality of needle insertions at different times and/or in different areas of the tumor, in order to increase the volume of the tumor covered by the injected mixture and/or to treat tumor cells that were not exposed to enough radiation in previous injections. Alternatively, the mixture is smeared or spread on a surface of tumor and/or on a surface of a cavity from which a tumor was removed by surgery.

Alternatively to injecting to the tumor, the mixture is injected to the patient's blood circulation, for example when the mixture includes targeted small particles.

In some embodiments, the mixture is injected to a vasculature next to the tumor in a manner which blocks and/or closes small blood vessels near or in the tumor. In embodiments using targeted nanoparticles, the mixture may additionally or alternatively be administered intravenously.

As discussed above, not all embodiments involve injecting (210) calcium. In some embodiments in which calcium is injected (210), the calcium is injected to the tumor before, during and/or after injection of the mixture through a separate needle from the needle used for injection (208) of the mixture. In some embodiments, the calcium is injected (210) concurrently with the mixture, for example using a split needle. Alternatively, the mixture and the calcium are loaded into the same needle and are injected one after the other into the tumor. Optionally, the calcium is loaded to the needle more distally and is injected first into the tumor. Alternatively, the mixture is loaded to the needle more distally and is injected before the calcium.

Injecting the calcium before the mixture increases the percentage of the mixture that turns into a gel before it leaves the tumor, as the calcium required to induce the gelation is already in the tumor when the mixture is injected. Concurrent injection of the mixture and the radium allows simpler operation by the medical practitioner performing the injection, while inducing immediate mixing of the agent in the mixture and the calcium.

In the above description, the agent and the radium are mixed together into the injected solution, before the injection. In other embodiments, a solution including the agent is injected to the tumor separately from administration of the radium radionuclides. In some of these embodiments, the radium radionuclides are administered by injecting a radium solution into the tumor. Alternatively, the radium radionuclides are administered by insertion of one or more sources which carry free radium into the tumor. The one or more sources are optionally designed to allow the free radium to leave the sources, after implantation of the one or more sources in the tumor. The sources, optionally, are biodegradable after implantation, so that the free radium is released into the tumor. Alternatively, the radium on the sources is attached loosely so that the radium is released from the source upon contact with tumor tissue and/or due to the temperature in the tumor. The radium released from the seeds is caught by the solution including the agent, and is thus prevented from escaping the tumor.

The separate injection allows producing the agent solution a longer time before the treatment and storing the agent solution for longer periods. In some embodiments, the agent solution is injected before the radium is administered, for example at least 5 seconds, at least 15 seconds, at least 45 seconds or even at least 90 seconds before the administration of the radium to the tumor. The early injection of the agent allows the agent time to settle in the tumor, before the radium is administered. Optionally, however, the radium is administered within less than 1 hour, less than 20 minutes or even less than 10 minutes from the injection of the agent solution. Alternatively, the agent solution is injected after administering radium to the tumor. Optionally, in this alternative, the agent solution is injected within less than 30 seconds, less than 15 seconds or even less than 5 seconds from the administering of the radium, so that the radium does not escape before the agent solution which provides long term fixation of the radium in the tumor is injected. In some embodiments administering the radium by injecting a radium solution, the radium solution and the agent solution are injected using a same needle. Alternatively, the radium solution and the agent solution are injected from different needles. In other embodiments, the separate injection of the radium solution and the agent solution is performed concurrently from two different needles.

In the method of FIG. 2, further injection (208) of the mixture is performed responsive to analysis (212) of medical images of the tumor. The acquiring of the images may be performed immediately after the injection and/or at later times, for example after several days (e.g., every 2 days or every 3 days). Alternatively or additionally, repeated injection may be performed periodically based on a predesigned plan. For example, repeated injections may be performed every two days, every week or every two weeks. In some embodiments, when treating a patient with multiple tumors, the mixture is injected to a plurality of separate tumors of the patient in a single treatment session. Alternatively, the mixture is injected to each tumor at a separate time, so as not to introduce large levels of radioactivity concurrently.

In other embodiments, rather than injecting the agent solution it is smeared on a surface requiring treatment, such as an external skin cancer tumor or tissue of a cavity after removal of a tumor. In some of these embodiments, a protective sheet may be placed on the smeared mixture in order to prevent damage to healthy tissue.

### CONCLUSION

While the above description relates to a mixture of an agent which turns into a hydrogel by addition of calcium ions with alpha emitter radium isotopes, it is noted that the agent could be used with proper adaptations also with betta emitter radium, such as radium-225. Furthermore, the agent could be used to form a radiotherapy mixture with radionuclides of other isotopes of biocompatible elements which transform the agent into a gel, or otherwise bond to the agent, and have a half-life suitable for use in medical radiotherapy. Elements that transform the agent into a gel could be any of those described in the above mentioned article: Hu et al., “Ions-induced gelation of alginate: Mechanisms and Applications”, International Journal of Biological Macromolecules, including Ba²⁺, Cu²⁺, Sr²⁺, Fe²⁺, Zn²⁺, Mn²⁺, A1³⁺ and Fe³⁺ and elements having similar properties, such as elements in the columns of these elements in the periodic table. These other isotopes are optionally beta emitters, positron emitters and/or electron capturers. These other isotopes include, for example, isotopes of strontium (⁹⁰Sr and ⁸⁹Sr, ⁸⁵Sr), isotopes of calcium, copper-64 and/or gold-198.

It will be appreciated that the above-described methods and apparatus are to be interpreted as including apparatus for carrying out the methods and methods of using the apparatus. It should be understood that features and/or steps described with respect to one embodiment may sometimes be used with other embodiments and that not all embodiments of the invention have all of the features and/or steps shown in a particular figure or described with respect to one of the specific embodiments. Tasks are not necessarily performed in the exact order described.

It is noted that some of the above-described embodiments may include structure, acts or details of structures and acts that may not be essential to the invention and which are described as examples. Structure and acts described herein are replaceable by equivalents which perform the same function, even if the structure or acts are different, as known in the art. The embodiments described above are cited by way of example, and the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art. Therefore, the scope of the invention is limited only by the elements and limitations as used in the claims, wherein the terms “comprise,” “include,” “have” and their conjugates, shall mean, when used in the claims, “including but not necessarily limited to.”

1. A mixture for treating a tumor, comprising:
an agent which turns into a hydrogel by addition of calcium ions;
a vehicle carrying the agent in a manner allowing injection of the mixture into a patient; and
radium radionuclides bonded to the agent in a concentration sufficient to treat the tumor by radiotherapy.

2. The mixture as in claim 1, wherein the vehicle comprises an aqueous solution, and the agent is dispersed homogenously in the aqueous solution.

3. The mixture as in claim 1, further comprising a substance which regulates immune-checkpoints dispersed in the mixture.

4. The mixture as in claim 1, further comprising a contrast material.

5. The mixture as in claim 1, wherein the mixture is thermosensitive such that the viscosity of the mixture increases by at least a factor of two when its temperature increases from room temperature to body temperature.

6. The mixture as in claim 1, wherein the agent comprises alginate.

7. The mixture as in claim 1, wherein the agent comprises Pluronics.

8. The mixture as in claim 1, wherein the radium radionuclides are radium-224 radionuclides.

9. The mixture as in claim 1, wherein the mixture further comprises calcium.

10. The mixture as in claim 9, wherein the mixture comprises calcium at a concentration of between 1-10 millimolar.

11. The mixture as in claim 1, wherein the mixture does not bond to radon and lead.

12. The mixture as in claim 1, wherein the agent which turns into a hydrogel by addition of calcium ions is between 0.5-4% of the mixture.

13. The mixture as in claim 1, wherein the agent is included in small particles carried by the vehicle.

14. The mixture as in claim 1, wherein the small particles comprise a metallic core surrounded by the agent.

15. A method for treating a tumor, comprising:
injecting, into a patient, a mixture including an agent which turns into a hydrogel in contact with calcium ions; and
injecting into the patient radium radionuclides of an activity suitable for treatment of the tumor.

16. The method of claim 15, further comprising injecting calcium into the tumor.

17. The method of claim 16, wherein injecting the calcium into the tumor is performed before injecting the mixture.

18. The method of claim 16, wherein injecting the calcium into the tumor is performed after injecting the mixture into the tumor.

19. The method of claim 16, wherein injecting the calcium into the tumor comprises including calcium in the mixture and injecting the mixture into the tumor.

20. The method of claim 16, wherein injecting the calcium into the tumor comprises concurrently injecting the mixture and the calcium.

21. The method of claim 15, wherein injecting the radium radionuclides into the tumor comprises including radium in the mixture and injecting the mixture into the tumor.

22. The method of claim 15, wherein injecting the mixture into the patient comprises injecting the mixture into the tumor.

23. A method for generating a mixture for treating a tumor, comprising:
providing an inert excipient;
adding an agent which turns into a hydrogel in contact with calcium ions to the inert excipient; and
adding radium radionuclides in a concentration sufficient to treat the tumor by radiotherapy, to the inert excipient.

24. The method of claim 23, wherein adding the agent and the radium to the inert excipient comprises combining the agent and the radium before they are added to the inert excipient, and adding the combination of the agent and the radium together to the inert excipient.

25. The method of claim 23, wherein adding the agent and the radium to the inert excipient comprises adding the radium to the inert excipient only after the agent is added to the inert excipient.
